(19) United States
(12) Patent Application Publication
(10) Pub. No.: US 2012/0307208 A1
(43) Pub. Date: Dec. 6, 2012

(54) APPARATUS AND METHOD FOR EYE TRACKING

(75) Inventor: Jonathan Vernon Trousdale, Fort Lauderdale, FL (US)

(73) Assignee: ROGUE TECHNOLOGIES, INC., Fort Lauderdale, FL (US)

(21) Appl. No.: 13/151,060

(22) Filed: Jun. 1, 2011

(51) Int. Cl. A61B 3/113

(52) U.S. Cl. 351/210; 351/246

(57) ABSTRACT

A system is provided to determine the gaze of a user. The disclosed system uses an array of light detectors, each located proximate to a light emitter. Light from the light emitters enters the eye and is partially reflected in by the retina. Light returning from the eye is detected by the light detectors and used to determine the gaze of the user.

### TECHNICAL FIELD

The present invention relates to an apparatus and method for human-computer interaction, and more particularly the present invention relates to an apparatus and method for determining a gaze of a user.

### BACKGROUND

Interactions between humans and computers involve the output of information from a computer to a user, such as by way of a monitor and speakers, and the input of information from a user to a computer, such as by way of a mouse and keyboard. However, the use of a mouse and keyboard as input devices can be less than optimal in terms of speed, performance and convenience. Furthermore in other applications, such as automotive, smartphone or research applications, the use of a keyboard and/or mouse may not be desirable.

Eye tracking systems have been seen as a desirable form of user input for many years. Eye tracking systems generally attempt to provide an indication of the physical area commanding the visual attention of a user. Many available eye tracking systems utilize features of the eye, such as the center of the pupil, in combination with one or more reflections of a light source from the cornea of the eye to estimate eye gaze. For example, one such system is described in U.S. Pat. No. 7,572,008 to Elvesjo, et al. However, such systems may suffer from one or more of the following limitations as well as other limitations not listed: high expense; a need for significant computing power; a need for complex calibration; limited functional spatial range; limited temporal resolution; and/or insufficient accuracy.

### BRIEF DESCRIPTION OF THE DRAWINGS

The present invention will become more fully understood from the detailed description and the accompanying drawings, wherein:

FIG. 1 is a perspective view of a display screen incorporating an eye tracking system according to this disclosure;

FIG. 2 is a schematic drawing of an eye in relation to an element of the eye tracking system according to this disclosure; and

FIG. 3 is a schematic drawing of an eye in relation to a coordinate system employed in this disclosure.

### DETAILED DESCRIPTION

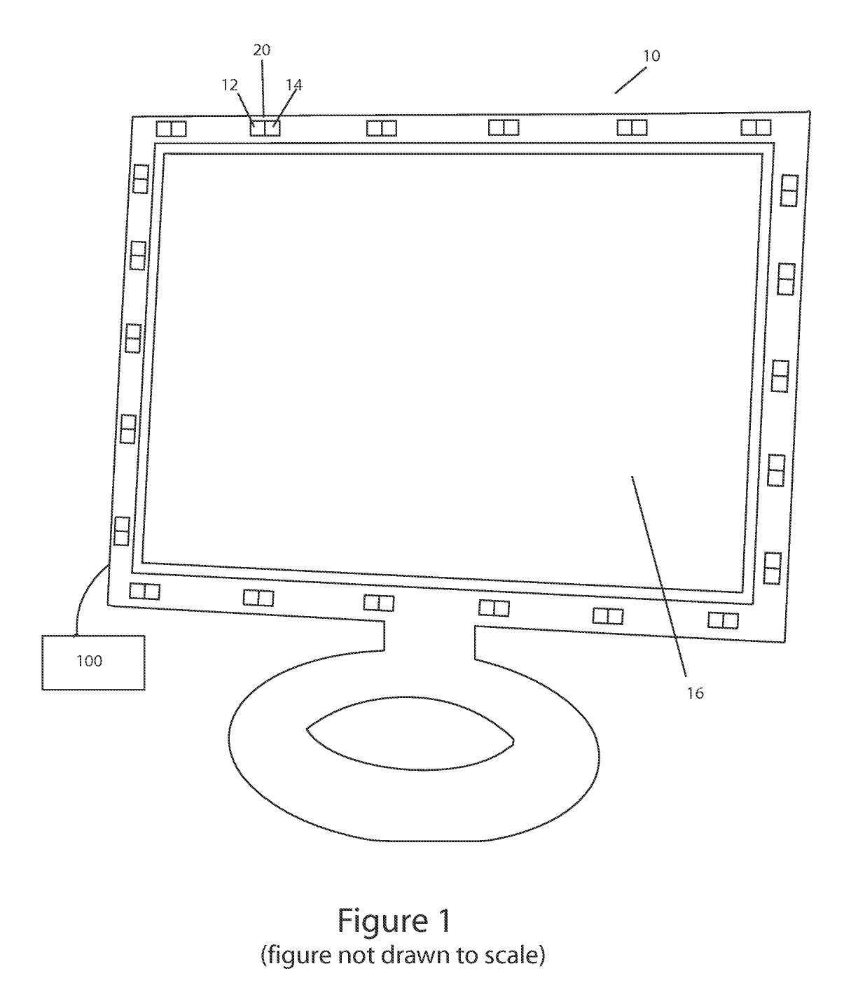

As illustrated in FIG. 1, an eye tracking system 10 is provided having an array of paired light emitters 12 and light detectors 14, each light emitter 12 being proximate to its paired light detector 14. As used herein a paired emitter 12 and detector 14 is referred to as an element 20. The embodiment illustrated in FIG. 1 uses a twenty-two element 20 array arranged around the periphery of a display screen 16; however, a wide range in both the number of elements 20 and the arrangement of elements 20 may be utilized.

In the disclosed embodiment, an infrared light emitting diode (LED), or a group of LEDs may be used as an emitter 12 and a spectrally matched photodiode may be used as a detector 14; however, a wide variety of emitters 12 and detectors 14 may alternately be utilized.

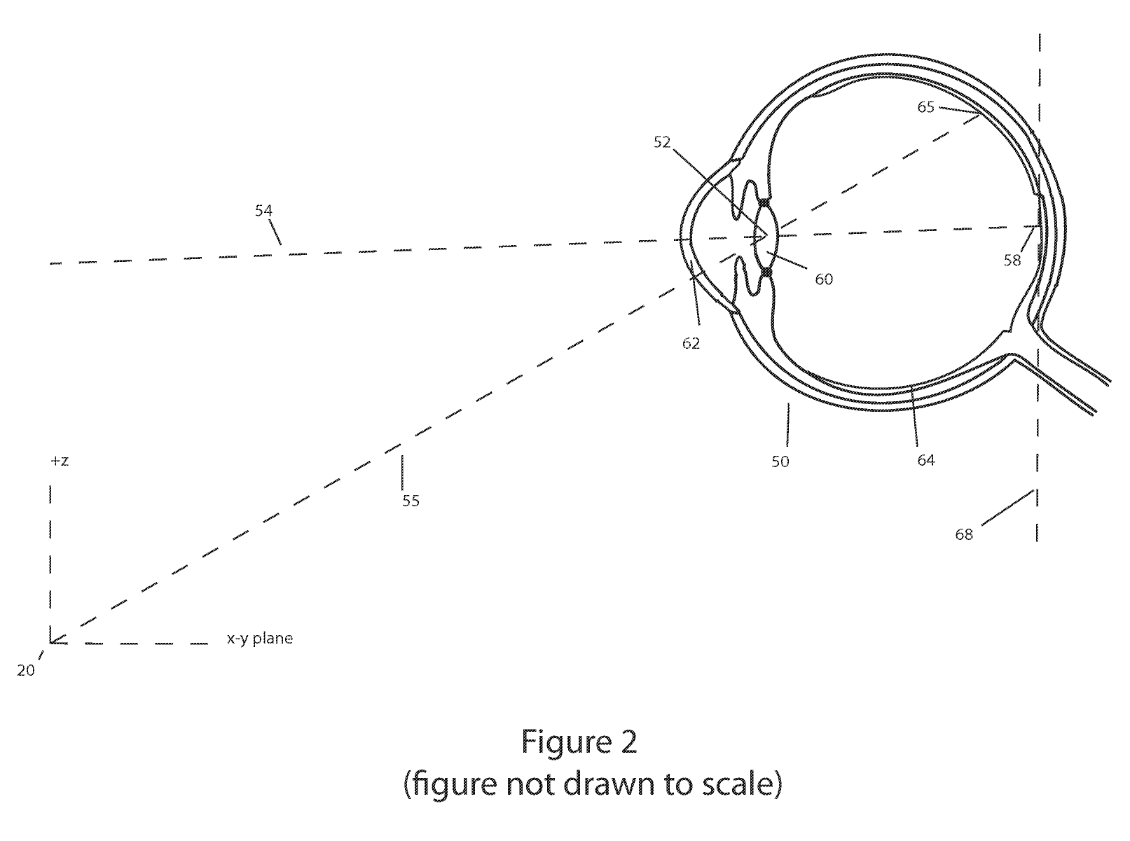

FIG. 2 illustrates the interaction of a single element 20 with a single eye 50. Light from emitter 12 may be emitted according to a known directivity. For example, an LED having a substantially radially symmetric directivity, and varying linearly from a maximum radiant intensity (I_{θ}=1.0 at θ=0° to zero radiant intensity (I_{θ}=0) at θ=90. Accordingly, the radiant intensity in the illustrated embodiment can be described as a function of θ according to Equation 1:

I=Iₘₐₓ(1−θ/π),θε[0,π] Eq. 1:

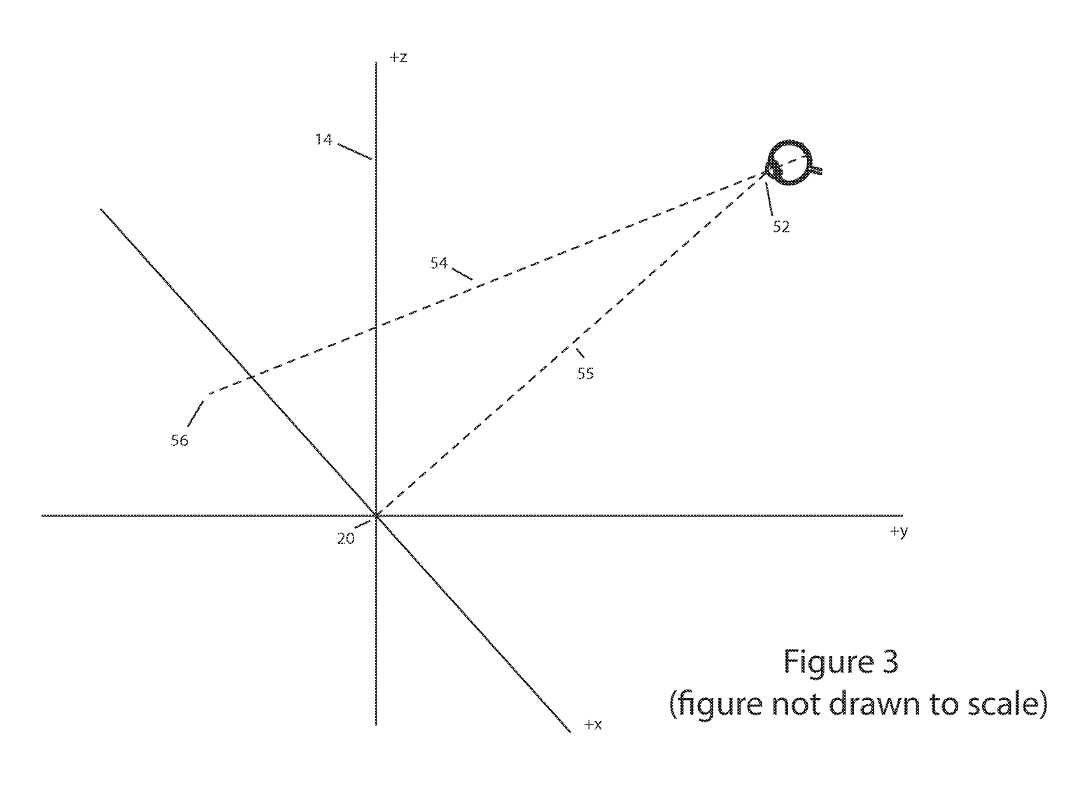

FIG. 3 illustrates the coordinate conventions used herein. In the illustrated embodiment, the array of elements 20 is arranged in the x-y plane, and the emitter 12 of the particular element 20 detailed in FIG. 2 has an optical axis substantially aligned with the z-axis. The x-axis and the y-axis may be aligned such that, for example, they are parallel with two substantially perpendicular sides of the eye tracking system 10, or the associated display 16, or as otherwise may be convenient.

As illustrated in FIG. 3, the vector \vec{R} 55 represents the vector from the emitter 12 at the origin of the coordinate system to the eye 50 at point E=(xᵣ,yᵣ,zᵣ). Thus, according to Equations 2 and 3 vector k and unit vector R are characterized, respectively, by Equation 2 and Equation 3:

$$\begin{matrix} \vec{R} = ( {x}_{r} , {y}_{r} , {z}_{r} ) & Eq . 2 \\ \hat{R} = ( \frac{{x}_{r}}{\sqrt{{x}_{r}^{2} + {y}_{r}^{2} + {z}_{r}^{2}}} , \frac{{y}_{r}}{\sqrt{{x}_{r}^{2} + {y}_{r}^{2} + {z}_{r}^{2}}} , \frac{{z}_{r}}{\sqrt{{x}_{r}^{2} + {y}_{r}^{2} + {z}_{r}^{2}}} ) = ( {\hat{R}}_{x} , {\hat{R}}_{y} , {\hat{R}}_{z} ) & Eq . 3\end{matrix}$$

Point E may generally represent the pupil 52 of the eye 50, or more particularly the center of the pupil 52 of the eye, or another feature of the eye 50, such as an optical center of the compound lens formed by the cornea 62 and the lens 60, as may be convenient. The angle θ represents the angle between \vec{R} 55 and the z-axis. This coordinate system is used herein for convenience; however, any of a variety of coordinate systems may be utilized.

As further illustrated in FIG. 3, a visual axis of the eye 50 is substantially collinear with a vector \vec{G} 54, and intersects the x-y plane at point G=(x_{g},y_{g},0) 56. Referring to FIG. 2, vector \vec{G} 54 and unit vector Ĝ are respectively characterized by Equation 4 and Equation 5:

$$\begin{matrix} \vec{G} = ( {x}_{r} - {x}_{g} , {y}_{r} - {y}_{g} , {z}_{r} ) & Eq . 4 \\ \hat{G} = ( \frac{{x}_{r} - {x}_{g}}{{( {x}_{r} - {x}_{g} )}^{2} + {( {y}_{r} - {y}_{g} )}^{2} + {z}_{r}^{2}} , \frac{{y}_{r} - {y}_{g}}{{( {x}_{r} - {x}_{g} )}^{2} + {( {y}_{r} - {y}_{g} )}^{2} + {z}_{r}^{2}} , \frac{{z}_{r}}{{( {x}_{r} - {x}_{g} )}^{2} + {( {y}_{r} - {y}_{g} )}^{2} + {z}_{r}^{2}} ) = … ( {\hat{G}}_{x} , {\hat{G}}_{y} , {\hat{G}}_{z} ) & Eq . 5\end{matrix}$$

Thus, the vector \vec{G} 54 is substantially collinear with a line passing from point G 56 to the fovea centralis 58 of the eye 50; wherein the point G 56 is an estimation for the location commanding the visual attention of a user.

With reference to FIGS. 2 and 3, for the sake of providing a simplified illustration, the eye 50 is modeled as a simple lens 60 system having a focal length d, with the retina 64 modeled as a portion of a sphere having radius r and centered at a point C=(x_{c},y_{c},z_{c}). The center of the lens 60 is positioned at distance d from the fovea centralis 58 along \vec{G} 54. Multiple simplifications are made to the actual structure of the eye 50 for this model; however, a more complex model may be used to improve the accuracy of the model. Accordingly the coordinates of point C may be defined as:

x_{c}=xᵣ+(d−r)Ĝₓ Eq. 6:

y_{c}=yᵣ+(d−r)Ĝ_{y} Eq. 7:

z_{c}=zᵣ+(d−r)Ĝ_{z} Eq. 8:

It follows that the equation for a sphere representing the retina is:

(x−x_{c})²+(y−y_{c})²+(z−z_{c})²=r² Eq. 9:

Furthermore, the fovea centralis 58 can be represented as point F=(x_{f}, y_{f}, z_{f}), where:

x_{f}=xᵣ+d*Ĝₓ Eq. 10:

y_{f}=y_{f}+d*Ĝ_{y} Eq. 11:

z_{f}=zᵣ+d*Ĝ_{z} Eq. 12:

According to this model, Ĝ is normal to the focal plane 68 of the lens 60 and includes point F. As such, the focal plane 68 may be characterized by Equation 13:

Ĝₓ(x−x_{f})+Ĝ_{y}(y−y_{f})+Ĝ_{z}(z−z_{f})=0 Eq. 13:

With the model constructed as such, it is possible to determine the intersection of the vector \vec{R} 55, and the retina 64 at a point K 65 by introducing a parameter t, such that:

K=E+\hat{R}t=(Xₖ,yₖ,zₖ) Eq. 14:

Where t is found as the solution to Equation 15, which is derived from Equations 6-9 and 14:

[\hat{R}ₓt+(d−r)Ĝₓ]²+[\hat{R}_{y}t+(d−r)Ĝ_{y}]²+[\hat{R}_{z}t+(d−r)Ĝ_{z}]²=r²,tεR,t>0 Eq. 15:

A vector from point K 65 to the focal plane 68, i.e. vector \vec{K}, is parallel to Ĝ, and can thus be described as:

\vec{K}=Ĝt₂ Eq. 16:

In Equation 16, t₂ is a second parameter, which in this case represents the distance from point K 65 to the focal plane 68. The value for parameter t₂ can be found from Equation 17, which simplifies to Equation 18:

Ĝₓ(xₖ+Ĝₓt₂−x_{f})+Ĝ_{y}(yₖ+Ĝ_{y}t₂−y_{f})+Ĝ_{z}(zₖ+Ĝd_{z}t₂−z_{f})=0 Eq. 17:

t₂=Ĝₓ(x_{f}−xₖ)+Ĝ_{y}(y_{f}−yₖ)+Ĝ_{z}(z_{f}−zₖ) Eq. 18:

With reference to FIG. 2, light leaving the emitter 12, enters the pupil 52 having an opening with area A. With reference to FIG. 2, the pupil 52 having opening area A, occupies a solid angle Ωᵢ in steradians according to Equation 19:

$${Ω}_{i} = \frac{A ( \hat{G} \cdot \hat{R} )}{{x}_{r}^{2} + {y}_{r}^{2} + {z}_{r}^{2}} \qquad Eq . 19$$

Accordingly the flux entering the eye, denoted by Φᵢ, from the emitter 12 can be estimated by Equation 20:

$${Φ}_{i} = {I}_{i} {Ω}_{i} = {I}_{max} ( 1 - \frac{{cos}^{- 1} ( \frac{{z}_{r}}{\sqrt{{x}_{r}^{2} + {y}_{r}^{2} + {z}_{r}^{2}}} )}{π} ) \frac{A ( \hat{G} \cdot \hat{R} )}{{x}_{r}^{2} + {y}_{r}^{2} + {z}_{r}^{2}} \qquad Eq . 20$$

According to the present model, light striking the retina 64 is diffusely reflected according to a reflectance value of the retina 64, denoted as Jₖ, which may vary depending on the location of K 65 on the retina 64. As an additional simplification to the model, light originating from emitter 12 and reflected from the retina 64 is assumed to be reflected from point K 65; however, light could be modeled to be reflected from a region surrounding point K 65 for increased accuracy. Thus, neglecting transmission and reflection losses between the front surface of the cornea and the retina for the purposes of the model, the light diffusely reflected at point K 65, denoted by Φᵣ, is estimated by Equation 21, where Jₖ is the reflectance of the retina at point K 65:

Φᵣ=JₖΦᵢ Eq. 21:

As the model assumes diffuse reflection, the intensity of light reflected by the retina 64, denoted by Iᵣ, is estimated by Equation 22:

$${I}_{r} = \frac{{φ}_{r}}{2 π} \qquad Eq . 22$$

Incorporating t from Equation 14, the flux leaving the eye 50 (Φₒ) is estimated by:

Φₒ=IᵣA(Ĝ·\hat{R})t⁻² Eq. 23:

As point K 65 is between the lens 60 and the focal plane 68, light reflected from point K 65 diverges as it leaves the lens 60, creating a virtual image of K at a distance s from the lens. With the focal length of lens 60 presumed according to the model to equal d, the distance s may be estimated according to a thin lens equation:

1/(−s)+1/(d−t₂)=1/d Eq. 24:

s=d(d/t₂−1) Eq. 25:

The value (−s) is used in Equation 24, to provide a positive value for s in Equation 25 because s represents the position of a virtual image.

As such, light reflected from K leaves the eye with an effective solid angle Ωₑ estimated by:

Ωₑ=A(Ĝ·\hat{R})/[s(Ĝ·\hat{R})]²=A(Ĝ·\hat{R})⁻¹s⁻² Eq. 24:

Thus the radiant intensity of light reflected from point K 65 through the pupil, denoted by Iₒ, is estimated by:

Iₒ=Φₒ/Ωₑ=Iᵣs²t⁻²(Ĝ·\hat{R})² Eq. 25:

Accordingly, the flux striking the detector, denoted by Φ_{d}, having an active area A_{d}, and aligned such that the z-axis is substantially normal to the active area, can be estimated by:

$${Φ}_{d} = {I}_{o} {\hat{R}}_{z} {A}_{d} / {[ \vert \vec{R} \vert + \frac{s}{( \hat{G} \cdot \hat{R} )} ]}^{2} \qquad Eq . 26$$

Accordingly, a signal read from the detector 14 is indicative of Φ_{d}. In the illustrated embodiment, detector 14 is a photodiode and either a voltage signal or a current signal from the photodiode may be indicative of Φ_{d}.

Thus, as shown in the equations above, Φ_{d} can be represented in terms of: Jₖ, Iₘₐₓ, A, A_{d}, d, r, x_{g}, y_{g}, xᵣ, yᵣ, and Zᵣ. The value of some of these variables can be estimated and provided as constants, or provided as a map (for example in the case of Jₖ). However, unless the position of the head is otherwise known, x_{g}, y_{g}, xᵣ, yᵣ, and zᵣ will likely need to be determined. One method to determine these values is to provide a sufficient number of elements 20 in the array to solve for the variables simultaneously.

In the case of the term Jₖ, which may vary with the position of point K, and thus may vary from element 20 to element 20, as different elements 20 may produce light that strikes the retina 64 at different points on the retina 64. Various methods may be used to determine Jₖ. One such method would be to use elements 20 having a detector 14 and more than one emitter 12, spaced in relatively close proximity to each other and the detector 14. The slight change in position of the emitters 12 will result in a slight change in the values of xᵣ and yᵣ, however, Jₖ may be estimated to be substantially constant because light from the close emitters 12 will impinge in proximate areas of the retina 64.

For example, if three emitters 12 are utilized with one emitter 12 in a central position, one emitter 12 aligned along the x-axis with the central emitter 12 and one emitter 12 aligned along the y-axis with the central emitter 12, values for

$$\frac{\partial {Φ}_{d}}{\partial {x}_{r}} and \frac{\partial {Φ}_{d}}{\partial {y}_{r}}$$

may be approximated and may be relatively insensitive to variations in Jₖ. According to this method, the partial derivatives of Φ_{d}, as provided in Equation 26, with respect to xᵣ and yᵣ may be utilized to find the solution for x_{g} and y_{g}.

In yet another alternative method, signals may be sampled at a high speed and

$$\frac{\partial {Φ}_{d}^{2}}{\partial {x}_{r} \partial t} and \frac{\partial {Φ}_{d}^{2}}{\partial {y}_{r} \partial t}$$

may be utilized to determine values for

$$\frac{\partial {x}_{g}}{\partial t} and \frac{\partial {y}_{g}}{\partial t} .$$

In this manner the velocity of the user's gaze may be approximated. By determining the velocity of a user's gaze, it may be possible to determine the current state of the user's gaze, i.e. fixation, saccade or smooth pursuit. Furthermore, because saccades may have substantially symmetrical velocity profiles, it may be possible to estimate the end point of a saccade, i.e. the user's next fixation point, immediately after the midpoint of a saccade. In this manner, a computer may estimate where a user will be looking before the user looks there to provide a highly responsive, and potentially anticipatory, user interface.

Another challenge in implementing this system is to eliminate effects of noise, i.e. sources of light from other sources striking the detector 14. One method to account for noise is to watch for a blinking action by the user, characterized by a drop in the signal output of the detectors 14 following a predictable pattern. When the pupil 52 is fully covered by the eyelid during a blink, the drop in the signal should achieve a local minimum. This local minimum approximates the signal caused by all sources of light, other than light reflected from the retina 64 and through pupil 52, which can be used as an indication of the signal attributable to noise. This noise signal can then be subtracted from further signals until the next blink event, at which time a new noise signal can be acquired.

Another method for accounting for noise is to use a band-pass filter to isolate signals having a frequency expected from microsaccades. As such, when the user is fixated and the eyes 50 of the user are engaged in microsaccades, much of the signal not resulting from light reflected by the retina 64 through the pupil 52 may be filtered out.

Another use of the impact of microsaccades would be to separate the signals attributable to the left eye and the right eye, and potentially eyes of multiple users. At any given time, each eye 50 may be engaged in a microsaccade having a different timing and a different vector than any other eye 50. As such, by separating signals having distinct vectors and timing, it may be possible to separate signals from distinct eyes 50.

Another method to reduce noise is to use detectors 14 sensitive only to a narrow frequency band that is matched to the frequency band of light emitted by the emitters 12. Alternatively and in a similar manner, a light filter passing only a narrow frequency band matched to the frequency band of light emitted by the emitter 12 may be utilized.

In yet another method to reduce noise, the emitters 12 may be pulsed such that no two emitters 12 are emitting light at the same time. According to this method, the signal of a given detector 14 is measured only when its corresponding emitter 12 is being pulsed.

The calculations discussed herein may be performed by a controller 100, as illustrated in FIG. 1, which may be located either internally or externally to display 16. The controller 100 may be connected to the elements 20 and may perform the calculations needed to estimate user gaze based on signals produced by the elements 20. If the eye tracking system 10 is part of a device having a CPU and associated components, the controller 100 may incorporate and/or utilize the CPU and/or associated components in performing its calculations. The controller 100 may also output a signal indicative of the user gaze estimation to the CPU and/or associated components to be utilized or stored by the device.

As various modifications could be made to the exemplary embodiments, as described above with reference to the corresponding illustrations, without departing from the scope of the invention, it is intended that all matter contained in the foregoing description and shown in the accompanying drawings shall be interpreted as illustrative rather than limiting. Thus, the breadth and scope of the present invention should not be limited by any of the above-described exemplary embodiments, but should be defined only in accordance with the following claims appended hereto and their equivalents.

What is claimed is:

1. An eye tracking system for estimating a visual gaze of a user comprising:
a sensor array having four or more elements, each element including a first light emitter and a light detector, wherein the light detectors are configured to generate signals indicative of the intensity of light emitted by the first light emitters and reflected from a plurality of surfaces, and
a controller configured to:
determine the portion of the signals attributable to light reflected by a retina of the user, and
estimate the visual gaze of the user based on the portion of the signals attributable to light reflected by the retina.

2. The eye tracking system of claim 1, wherein the first light emitters are light emitting diodes and the light detector is a photodiode.

3. The eye tracking system of claim 2, wherein the light emitting diodes are infrared light emitting diodes.

4. The eye tracking system of claim 1, wherein the controller determines the portion of the signal attributable to light reflected by the retina by passing the signal through a band-pass filter.

5. The eye tracking system of claim 4, wherein the band-pass filter is configured to pass frequencies corresponding to expected microsaccadic frequencies of the user.

6. The eye tracking system of claim 1, wherein each element further includes a second light emitter proximate to the light detector and a third light emitter proximate to the light detector.

7. The eye tracking system of claim 6, wherein each respective set of first, second and third light emitters form a substantially right angle.

8. The eye tracking system of claim 1, wherein each first light emitter is pulsed on and off.

9. The eye tracking system of claim 8, wherein only one first light emitter is pulsed on at a given time.

10. The eye tracking system of claim 8, wherein the signal generated by a respective light detector when the respective light detector's first light emitter is off is disregarded by the controller in the estimation of eye gaze.

11. A method for estimating a visual gaze of a user comprising the steps of:
providing a light emitter;
providing a light detector proximate to the light emitter;
emitting light from the light emitter;
detecting the emitted light reflected from a plurality of surfaces;
determining the portion of the detected emitted light that was reflected from a retina of the user; and
estimating the visual gaze of the user based on the portion of the detected emitted light that was reflected from the retina of the user.

12. The method of claim 11 further comprising the step of pulsing the light emitter on and off.

13. The method of claim 11 further including the step of generating a signal indicative of the detected emitted light.

14. The method of claim 13, wherein the step of determining the portion of the detected emitted light that was reflected from a retina of the user includes the step of isolating a frequency band in the signal.

15. The method of claim 11 further comprising the step of estimating the visual gaze of the user at a first time and estimating the visual gaze of the user at a second time.

16. The method of claim 15 further comprising the step of estimating a first velocity of the visual gaze of the user based on the estimation of the visual gaze of the user at the first time and the visual gaze of the user at the second time.

17. The method of claim 16 further comprising the step of estimating a second velocity of the visual gaze of the user based on the estimation of the visual gaze of the user at a third time and the visual gaze of the user at a fourth time.

18. The method of claim 17 further comprising the step of determining the visual state of the user based on the estimated first and second velocities.

19. The method of claim 17 further comprising the step of estimating an end point of a saccade based on the estimated first and second velocities.

20. An eye tracking system for estimating a visual gaze of a user comprising:
a first light emitter,
a second light emitter,
a light detector disposed proximate to the light emitter wherein the light detector is configured to generate a signal indicative of the intensity of light emitted by the light emitters and reflected from a plurality of surfaces, and
a controller configured to:
pulse the first light emitter on and off,
pulse the second light emitter on and off,
compare the signal at a first time when the first light emitter is on and the second light emitter is off to the signal at the second time when the first light emitter is off and the second light emitter is on, and
estimate the visual gaze of the user based on the comparison of the signal at the first time and the signal at the second time.
